(19) United States
(12) Patent Application Publication
(10) Pub. No.: US 2015/0320869 A1
(43) Pub. Date: Nov. 12, 2015

(54) COMPOSITION COMPRISING AN ORGANIC LIQUID DILUENT AND A SPECIFIC HYDROXYPROPYL METHYLCELLULOSE

(71) Applicant: Dow Global Technologies LLC, Midland, MI (US)

(72) Inventors: Meinolf Brackhagen, Bomlitz (DE); Robert L. Schmitt, Annandale, NJ (US); Matthias Knarr, Bomlitz (DE)

(21) Appl. No.: 14/804,489

(22) Filed: Jul. 21, 2015

Related U.S. Application Data
(62) Division of application No. 13/942,775, filed on Jul. 16, 2013.
(60) Provisional application No. 61/672,412, filed on Jul. 17, 2012.

(51) Int. Cl. A61K 47/38; A61K 9/50

(52) U.S. Cl. CPC A61K 47/38; A61K 9/5047; A61K 9/5089

(57) ABSTRACT

A liquid composition which comprises an organic liquid diluent and at least one hydroxypropyl methylcellulose having 28 to 30 weight percent of methoxyl groups, 7 to 12 weight percent of hydroxypropoxyl groups and a sum of methoxyl groups and hydroxypropoxyl groups of from 38.5 to 42 weight percent is stable over an extended time period.

The liquid composition is useful for preparing a solid dispersion comprising at least one active ingredient in at least one hydroxypropyl methylcellulose by spray-drying.

Alternatively a solid dispersion can be produced by blending and extruding at least one active ingredient, at least one hydroxypropyl methylcellulose described above and optionally one or more adjuvants.

### FIELD

This invention relates to a liquid composition comprising an organic liquid diluent and a specific hydroxypropyl methylcellulose and to a solid dispersions comprising an active ingredient in a hydroxypropyl methylcellulose.

### INTRODUCTION

A large number of presently known drugs have a low solubility in water, and thus complex techniques are required to prepare a suitable dosage form. Much research is spent on the use of pharmaceutically acceptable water-soluble polymers in combination with drugs of low water solubility. The use of water-soluble polymers aims at reducing the crystallinity of the drug, thereby minimizing the activation energy necessary for the dissolution of the drug, as well as establishing hydrophilic conditions around the drug molecules, thereby improving the solubility of the drug itself to increase its bioavailability, i.e., its in vivo absorption by an individual upon administration. However, simple blending of a water-soluble polymer with a drug of low water solubility generally does not reduce the crystallinity of the drug nor generally improve said drug's solubility.

G. Van den Mooter, “The use of amorphous solid dispersions: A formulation strategy to overcome poor solubility and dissolution rate”, Drug Discov Today: Technol (2011), doi:10.1016/j.ddtec.2011.10.002, discusses the preparation of amorphous solid dispersions to increase the bioavailability of poorly soluble drugs by improving their rate and extent of dissolution. The two most applied manufacturing methods for preparing amorphous solid dispersions are said to be spray drying and hot melt extrusion. The former process starts from a solution of the drug and a carrier in a common organic solvent or mixture of aqueous and organic solvents. This solution is atomized using a nozzle and the solvent is subsequently quickly evaporated (order of magnitude is milliseconds). The very fast solvent evaporation contributes to the amorphous state of the solid dispersion.

Dallas B. Warren et al. (Journal of Drug Targeting, 2010; 18(10): 704-731) have studied the use of water-soluble cellulose ethers as polymeric precipitation inhibitors, such as carboxymethyl cellulose (CMC), methyl cellulose (MC), hydroxyethyl cellulose (HEC), and hydroxypropylmethyl cellulose (HPMC) to improve the absorption of poorly water-soluble drugs.

S.L. Raghavan et al. (International Journal of Pharmaceutics 212 (2001) 213-221), have studied the influence of HPMC, MC, polyvinyl pyrrolidone (PVP) and polyethylene glycol (PEG400) on the crystallization of hydrocortisone acetate (HA).

International Patent Application W02008/047201 discloses solid dispersions which comprise a poorly water soluble ionizable drug, a cationic species, and a dispersion polymer, such as hydroxypropyl methylcellulose (HPMC). According to the examples a drug and HPMC (E3 Prem LV; Methocel®, available from The Dow Chemical Company, Midland, Mich.) are mixed with water and methanol to form spray solutions. Solid spray-dried dispersions of the drug in HPMC are produced from this solution.

Unfortunately, compositions comprising an organic liquid diluent and a cellulose ether, such as hydroxypropyl methylcellulose, often are not storage stable but exhibit a huge viscosity increase after storage of the liquid composition over an extended time period. The viscosity increase can often be avoided by storing the liquid composition below room temperature, but this is often undesirable since it complicates storage and adds to storage costs. Moreover, the observed viscosity increase often limits the achievable content of the cellulose ether in the liquid composition, thus adding transportation and solvent recovery costs.

In view of the high importance and large number of poorly water soluble drugs, it is an object of the present invention to provide new liquid compositions which comprise an organic liquid diluent and a cellulose ether into which active ingredients can be incorporated, such as poorly water-soluble drugs, and which can be spray-dried to produce solid dispersions comprising the active ingredient in a cellulose ether. A preferred object of the present invention is to provide new liquid compositions comprising an organic liquid diluent and a cellulose ether which are more storage stable than known comparable liquid compositions comprising an organic liquid diluent and at a cellulose ether.

### Summary

Surprisingly, it has been found that the storage stability of liquid compositions comprising an organic liquid diluent and a hydroxypropyl methylcellulose can be increased if a hydroxypropyl methylcellulose of a very specific percentage of methoxyl groups, hydroxypropoxyl groups and sum of methoxyl groups and hydroxypropoxyl groups is incorporated into the liquid composition.

Accordingly, one aspect of the present invention is a liquid composition which comprises an organic liquid diluent and at least one hydroxypropyl methylcellulose having 28 to 30 weight percent of methoxyl groups, 7 to 12 weight percent of hydroxypropoxyl groups and a sum of methoxyl groups and hydroxypropoxyl groups of from 38.5 to 42 weight percent.

Another aspect of the present invention is the use of the liquid composition as defined above for preparing a solid dispersion comprising at least one active ingredient in at least one hydroxypropyl methylcellulose.

Yet another aspect of the present invention is a solid dispersion comprising at least one active ingredient in at least one hydroxypropyl methylcellulose, wherein the hydroxypropyl methylcellulose has 28 to 30 weight percent of methoxyl groups, 7 to 12 weight percent of hydroxypropoxyl groups and a sum of methoxyl groups and hydroxypropoxyl groups of from 38.5 to 42 weight percent.

Yet another aspect of the present invention is a process for producing the solid dispersion as defined above which comprises the steps of blending a) at least one hydroxypropyl methylcellulose having 28 to 30 weight percent of methoxyl groups, 7 to 12 weight percent of hydroxypropoxyl groups and a sum of methoxyl groups and hydroxypropoxyl groups of from 38.5 to 42 weight percent, b) one or more active ingredients and c) one or more optional additives, and subjecting the blend to extrusion.

Yet another aspect of the present invention is a process for producing the solid dispersion as defined further above which comprises the steps of providing the liquid composition as defined above and removing liquid diluent from the liquid composition.

Yet another aspect of the present invention is a process for coating a dosage form which comprises the step of contacting the liquid composition as defined above with the dosage form.

Yet another aspect of the present invention is a process for the manufacture of capsules which comprises the step of contacting the liquid composition as defined above with dipping pins.

### DETAILED DESCRIPTION

The liquid composition of the present invention comprises at least one hydroxypropyl methylcellulose which has 28 to 30 weight percent of methoxyl groups, 7 to 12 weight percent of hydroxypropoxyl groups and a sum of methoxyl groups and hydroxypropoxyl groups of from 38.5 to 42 weight percent. The weight percentages are based on the total weight of the hydroxypropyl methylcellulose. The sum of the methoxyl groups and hydroxypropoxyl groups preferably is from 39.0 to 42 weight percent, more preferably from 39.3 to 42 weight percent, and most preferably from 39.5 to 41.5 weight percent. Preferably the hydroxypropyl methylcellulose has 28.8 to 30 weight percent of methoxyl groups. By convention, the weight percent is an average weight percentage based on the total weight of the cellulose repeat unit, including all substituents. The content of the methoxyl group is reported based on the mass of the methoxyl group (i.e., —OCH₃). The content of the hydroxypropoxyl group is reported based on the mass of the hydroxypropoxyl group (i.e., —O—CH₂CH(CH₃)—OH). The determination of the % methoxyl and % hydroxypropoxyl in hydroxypropyl methylcellulose (HPMC) is carried out according to the United States Pharmacopeia (USP 35, “Hypromellose”, pages 3467-3469). The procedure is described in more details in the Examples.

The hydroxypropyl methylcellulose incorporated in the liquid composition and the solid dispersion of the present invention generally has a viscosity of from 1.2 to 100 mPa's, preferably from 1.2 to 50 mPa's, more preferably from 1.2 to 10 mPa's, most preferably from 2.4 to 7 mPa's, and in particular from 4.0 to 7 mPa's, measured as a 2 wt.-% solution in water at 20° C. The 2% by weight cellulose ether solution in water is prepared according to United States Pharmacopeia (USP 35, “Hypromellose”, pages 3467-3469), followed by an Ubbelohde viscosity measurement according to DIN 51562-1:1999-01 (January 1999).

The composition of the present invention is liquid at 25 ° C. and atmospheric pressure and comprises an organic liquid diluent, in addition to at least one HPMC as described above. The term “organic liquid diluent” as used herein means an organic solvent or a mixture of two or more organic solvents that is liquid at 25 ° C. and atmospheric pressure. Preferred organic liquid diluents are polar organic solvents having one or more heteroatoms, such as oxygen, nitrogen or halogen like chlorine. More preferred organic liquid diluents are alcohols, for example multifunctional alcohols, such as glycerol, or preferably monofunctional alcohols, such as methanol, ethanol, isopropanol or n-propanol; ethers, such as tetrahydrofuran, ketones, such as acetone, methyl ethyl ketone, or methyl isobutyl ketone; acetates, such as ethyl acetate; halogenated hydrocarbons, such as methylene chloride; or nitriles, such as acetonitrile. More preferably the organic liquid diluents have 1 to 6, most preferably 1 to 4 carbon atoms. The liquid composition of the present invention may additionally comprise water; however, the liquid composition should comprise more than 50, more preferably at least 65, and most preferably at least 75 weight percent of an organic liquid diluent and less than 50, more preferably up to 35, and most preferably up to 25 weight percent of water, based on the total weight of the organic liquid diluent and water. Specific examples of preferred organic liquid diluents, optionally mixed with minor amounts of water are: methanol, tetrahydrofuran, methylene chloride, a blend of 80 to 95 weight percent of methanol and 20 to 5 weight percent of water, a blend of 80 to 95 weight percent of tetrahydrofuran and 20 to 5 weight percent of water, a blend of 55 to 85 weight percent of acetone and 45 to 15 weight percent of water, a blend of 15 to 85 weight percent of acetone and 85 to 15 weight percent of methanol, a blend of 15 to 85 weight percent of methyl ethyl ketone and 85 to 15 weight percent of methanol, a blend of 30 to 50 weight percent of acrylonitrile and 70 to 50 weight percent of a C₁₋₄-monoalcohol, such as methanol, ethanol, isopropylalcohol, or n-propanol; a blend of 30 to 50 weight percent of methanol and 70 to 50 weight percent of tetrahydrofuran or ethyl acetate, or a blend of 70 to 90 weight percent of ethanol and 10 to 30 weight percent of tetrahydrofuran or ethyl acetate.

The liquid composition of the present invention comprising an organic liquid diluent and an above-described HPMC has been found to be surprisingly stable upon storage. Surprisingly, it has been found that the liquid composition of the present invention is more storage stable and exhibits a smaller viscosity increase after storage of the liquid composition over an extended time period than a comparable liquid composition which comprises a hydroxypropyl methylcellulose which has 28 to 30 weight percent of methoxyl groups, 7 to 12 weight percent of hydroxypropoxyl groups and a sum of methoxyl groups and hydroxypropoxyl groups of less than 38.5 weight percent. When the liquid composition of the present invention comprises an organic liquid diluent and 10 weight percent of the above-described HPMC, based on the total weight of the liquid composition, its viscosity at 25 ° C. 30 minutes after its preparation typically is in the range of 100 to 50,000 mPa•s, more typically 200 to 20,000 mPa•s, most typically 500 to 10,000 mPa•s, measured as indicated above. When such liquid composition of the present invention, which comprises 10 weight percent of the above-described cellulose ether, is stored for at least 16 hours at 25° C., typically the viscosity of the liquid composition is not more than the 15-fold viscosity, more typically not more than the 10-fold viscosity of the liquid composition at 25° C. 30 minutes after the liquid composition has been prepared. Accordingly, the liquid composition of the present invention comprising an organic liquid diluent and an above-described HPMC does not tend to undesired viscosity increase upon storage at room temperature. The reduced tendency to viscosity increase allows a higher concentration of at least one above-described HPMC in a liquid composition comprising an organic liquid diluent while still preserving the flowability of the liquid composition. The increased storage stability is of particular importance if the composition of the present invention is directly used in liquid form, for example in the form of a suspension, a sprayable composition, or a syrup as described further below. However, the increased storage stability is also of high importance if the liquid diluent is removed from the liquid composition to produce various dosage forms as described further below. The increased storage stability increases the processing window, i.e., the possible time period from the preparation of the liquid composition until its further processing.

The liquid composition of the present invention is useful as an excipient system for active ingredients and particularly useful as an intermediate for preparing an excipient system for active ingredients, such as fertilizers, herbicides or pesticides, or biologically active ingredients, such as vitamins, herbals and mineral supplements and drugs.

Accordingly, the liquid composition of the present invention preferably comprises one or more active ingredients, most preferably one or more drugs. The term “drug” is conventional, denoting a compound having beneficial prophylactic and/or therapeutic properties when administered to an animal, especially humans. Preferably, the drug is a “low-solubility drug”, meaning that the drug has an aqueous solubility at physiologically relevant pH (e.g., pH 1-8) of about 0.5 mg/mL or less. The invention finds greater utility as the aqueous solubility of the drug decreases. Thus, compositions of the present invention are preferred for low-solubility drugs having an aqueous solubility of less than 0.1 mg/mL or less than 0.05 mg/mL or less than 0.02 mg/mL, or even less than 0.01 mg/mL where the aqueous solubility (mg/mL) is the minimum value observed in any physiologically relevant aqueous solution (e.g., those with pH values between 1 and 8) including USP simulated gastric and intestinal buffers.

The HPMC comprised in the liquid composition of the present invention and in the solid dispersion of the present invention is able to maintain the concentration of poorly water-soluble active ingredients, such as poorly water-soluble drugs in aqueous solutions at supersaturation levels. A considerably higher concentration of a poorly water-soluble active ingredient in an aqueous solution can be maintained than in the absence of a HPMC. The degree of supersaturation of a poorly water-soluble active ingredient in an aqueous solution depends on various factors, such as the physical stability and the dissolution rate of a given active ingredient. Dwayne T. Friesen et al. in MOLECULAR PHARMACEUTICS VOL. 5, NO. 6, 1003-1019, 2008 have classified compounds with a structurally diverse range of physicochemical properties on a physical property map Tm/Tg ratio versus log P. The log P value is a standard measure of the lipophilicity of a compound. Log P, defined as the base 10 logarithm of the ratio of (1) the drug concentration in an octanol phase to (2) the drug concentration in a water phase when the two phases are in equilibrium with each other, is a widely accepted measure of hydrophobicity. Log P may be measured experimentally or calculated using methods known in the art. When using a calculated value for Log P, the highest value calculated using any generally accepted method for calculating Log P is used. Calculated Log P values are often referred to by the calculation method, such as Clog P, Alog P, and Mlog P. The Log P may also be estimated using fragmentation methods, such as Crippen's fragmentation method (27 J.Chem.lnf.Comput.Sci. 2 1 (1987)); Viswanadhan's fragmentation method (29 J.Chem.lnf.Comput.Sci. 163 (1989)); or Broto's fragmentation method (19 Eur.J.Med.Chem.-Chim Theor. 7 1 (1984)).

$$log {P}_{oct / wat} = log ( \frac{{[ solute ]}_{octanol}}{{[ solute ]}_{water}^{un \text{-} ionized}} )$$

Compounds with high log P values are very hydrophobic and tend to have extremely low water solubilities (often less than 1 μg/mL when their melting points are above about 100° C.) and low propensities for wetting when placed into water.

Tm is the melting temperature and Tg is the glass transition temperature of the compound at atmospheric pressure. Dwayne T. Friesen et al. have divided the compounds into four groups based on their position on this physical property map TmITg ratio versus log P (FIG. 14 on page 1018 in MOLECULAR PHARMACEUTICS VOL. 5, NO. 6, 2008). The first group, Group 1, consists of compounds with relatively low Tm/Tg ratios (<1.25 K/K) and low to moderate log P values (less than about 6); Compounds in Group 2 have somewhat higher Tm/Tg ratios (1.25-1.4) and low to moderate log P values (less than about 6). Compounds in Group 3 have even higher Tm/Tg values (greater than 1.4) and low to moderate log P values (less than about 6). Finally, Group 4 compounds have high log P values (at least about 6).

A preferred aspect of the present invention is a liquid composition or a solid dispersion which comprises at least one HPMC as described above and additionally at least one active ingredient that has a Tm/Tg ratio of more than 1.0 up to 1.8, preferably more than 1.1 up to 1.6, more preferably from 1.15 to 1.5, most preferably from 1.25 to 1.40, wherein the melting temperature Tm and the glass transition temperature Tg each are in Kelvin. The active ingredient preferably has a log P of more than 1 up to 11, preferably 1.5 to 8, most preferably 2 to 6.

The active ingredient does not need to be a low-solubility active ingredient in order to benefit from this invention, although low-solubility active ingredients represent a preferred class for use with the invention. An active ingredient that exhibits appreciable aqueous solubility in the desired environment of use may have an aqueous solubility up to 1 to 2 mg/mL, or even as high as 20 to 40 mg/mL. Useful low-solubility drugs are listed in the International Patent Application WO 2005/115330, pages 17-22.

The liquid composition of the present invention preferably comprises from 1 to 40 weight percent, more preferably from 2.5 to 30 weight percent, most preferably from 5 to 25 weight percent, and particularly from 7 to 20 percent of at least one HPMC as described above, from 40 to 99 weight percent, more preferably from 54.9 to 97.4 weight percent, most preferably from 65 to 94.5 weight percent and particularly from 70 to 92 percent of i) an organic liquid diluent or ii) an organic liquid diluent blended with a minor amount of water, e.g. an amount of water described further above, and from 0 to 40 percent, preferably from 0.1 to 40 percent, most preferably from 0.5 to 25 percent, and particularly from 1 to 15 percent of an active ingredient, based on the total weight of the liquid composition.

In one aspect of the invention the liquid composition of the present invention comprising at least one HPMC as described above, one or more active ingredients and optionally one or more adjuvants can be used in liquid form, for example in the form of a suspension, a sprayable composition, or a syrup. The liquid composition is useful, e.g., for oral, ocular, topical, rectal or nasal applications. The liquid diluent should generally be pharmaceutically acceptable, such as ethanol or glycerol, optionally mixed with minor amounts of water as described above.

In another aspect of the invention the liquid composition of the present invention is used for producing a solid dispersion comprising at least one active ingredient, such as a drug described further above, in at least one HPMC as described above and optionally one or more adjuvants. The solid dispersion is produced by removing the liquid diluent from the composition. The liquid diluent is the liquid organic diluent, optionally blended with a minor amount of water as described above; i.e., when the composition comprises water as an optional additive, organic liquid diluent and water are removed from the liquid composition to prepare the solid dispersion of the present invention.

One method of removing the liquid diluent from the liquid composition is by casting the liquid composition into a film or a capsule or by applying the liquid composition onto a solid carrier that in turn may comprise an active ingredient. A preferred method of producing the solid dispersion is by spray-drying. The term “spray-drying” refers to processes involving breaking up liquid mixtures into small droplets (atomization) and rapidly removing solvent from the mixture in a spray-drying apparatus where there is a strong driving force for evaporation of solvent from the droplets. Spray-drying processes and spray-drying equipment are described generally in Perry's Chemical Engineers' Handbook, pages 20-54 to 20-57 (Sixth Edition 1984). More details on spray-drying processes and equipment are reviewed by Marshall, “Atomization and Spray-Drying,” 50

Chem. Eng. Prog. Monogr. Series 2 (1954), and Masters, Spray Drying Handbook (Fourth Edition 1985). A useful spray-drying process is described in the International Patent Application WO 2005/115330, page 34, line 7-page 35, line 25.

Alternatively, the solid dispersion of the present invention may be prepared by i) blending a) at least one HPMC defined above, b) one or more active ingredients and c) one or more optional additives, and ii) subjecting the blend to extrusion. The term “extrusion” as used herein includes processes known as injection molding, melt casting and compression molding. Techniques for extruding compositions comprising an active ingredient such as a drug are known and described by Joerg Breitenbach, Melt extrusion: from process to drug delivery technology, European Journal of Pharmaceutics and Biopharmaceutics 54 (2002) 107-117 or in European Patent Application EP 0 872 233. The above-mentioned components a), b) and optionally c) are preferably mixed in the form of particles, more preferably in powdered form. The components a), b) and optionally c) may be pre-mixed before feeding the blend into a device utilized for extrusion, preferably melt-extrusion. Useful devices for extrusion, specifically useful extruders, are known in the art. Alternatively, the components a), b) and optionally c) may be fed separately into the extruder and blended in the device before or during a heating step. Preferably components a), b) and optionally c) are pre-blended in an extruder feeder and fed from there into the extruder. The composition or the components that has or have been fed into an extruder are passed through a heated area of the extruder at a temperature which will melt or soften the composition or at least one or more components thereof to form a blend throughout which the active ingredient is dispersed. The blend is subjected to extrusion and caused to exit the extruder. Typical extrusion temperatures are from 50 to 210° C., preferably from 70 to 200° C., more preferably from 90 to 190° C., as determined by the setting for the extruder heating zone(s). An operating temperature range should be selected that will minimize the degradation or decomposition of the active ingredient and other components of the composition during processing. Single or multiple screw extruders, preferably twin screw extruders, can be used in the extrusion process of the present invention.

The molten or softened mixture obtained in the extruder are forced through one or more exit openings, such as one or more nozzles or dies. The molten or softened mixture then exits via a die or other such element having one or a plurality of openings, at which time, the extruded blend (now called the extrudate) begins to harden. Since the extrudate is still in a softened state upon exiting the die, the extrudate may be easily shaped, molded, chopped, spheronized into beads, cut into strands, tabletted or otherwise processed to the desired physical form. The extrudate can optionally be cooled to hardening and ground into a powdered form.

The solid dispersion of the present invention preferably comprises from 20 to 99.9 percent, more preferably from 30 to 98 percent, and most preferably from 60 to 95 percent of a HPMC a) as described above, and preferably from 0.1 to 80 percent, more preferably from 2 to 70 percent, and most preferably from 5 to 40 percent of an active ingredient b), based on the total weight of the HPMC a) and the active ingredient b). The combined amount of the HPMC a) and the active ingredient b) is preferably at least 70 percent, more preferably at least 80 percent, and most preferably at least 90 percent, based on the total weight of the solid dispersion. The remaining amount, if any, are one or more of the adjuvants c) as described below. The solid dispersion can comprise one or more of the HPMCs a), one or more of the active ingredients b), and optionally one or more of the adjuvants c), however their total amount is generally within the above-mentioned ranges.

Once the solid dispersion comprising at least one active ingredient in at least one HPMC has been formed, several processing operations can be used to facilitate incorporation of the dispersion into a dosage form. These processing operations include drying, granulation, and milling. The inclusion of optional adjuvants in the solid dispersion may be useful in order to formulate the composition into dosage forms, such as tablets, pills, granules, pellets, caplets microparticles, fillings of capsules, or into pastes, creams, suspensions or slurries. The amount of the active ingredient in the dosage form is generally is at least 0.1 percent, preferably at least 1 percent, more preferably at least 3 percent, most preferably at least 5 percent and generally up to 70 percent, or up to 50 percent, or up to 30 percent, or up to 25 percent, based on the total weight of the dosage form.

In another aspect of the invention the liquid composition of the present invention may be used for coating dosage forms, such as tablets, granules, pellets, caplets, lozenges, suppositories, pessaries or implantable dosage forms, to form a coated composition. If the liquid composition of the present invention comprises an active ingredient, such as a drug, drug layering can be achieved, i.e., the dosage form and the coating may comprise different active ingredients for different end-uses and/or having different release kinetics.

In yet another aspect of the invention the liquid composition of the present invention may be used for the manufacture of capsules in a process which comprises the step of contacting the liquid composition with dipping pins.

The liquid composition and the solid dispersion of the present invention may further comprise optional additives, such as coloring agents, pigments, opacifiers, flavor and taste improvers, antioxidants, plasticizers, surfactants, lubricants, anti-tack agents, glidants, fillers, disintegrants, binders, salts, such as sodium chloride; saccharides, such as white sugar and lactose; a second cellulose ether, and any combination thereof. Optional additives are preferably pharmaceutically acceptable. Useful amounts and types of one or more optional adjuvants are generally known in the art and depend on the intended end-use of the liquid composition or the solid dispersion of the present invention. A large variety of optional adjuvants is disclosed in International Patent Application WO 2005/115330, page 45, line 20-page 46, line 33.

The following examples are for illustrative purposes only and are not intended to limit the scope of the present invention. All percentages are by weight unless otherwise specified.

### Examples 1 and 2 and Comparative Examples A and B

The determination of the % methoxyl and % hydroxypropoxyl in hydroxypropyl methylcellulose (HPMC) was carried out according to the United States Pharmacopeia (USP 35, “Hypromellose”, pages 3467-3469) unless specified otherwise below:

Carrier gas: Helium

Detector: Hydrogen flame-ionization

Column: Fused-Silica capillary column, stationary phase 20% -Diphenyl-80%-Dimethyl-polysiloxan, e.g. RTX 20, length 30 m, i. d. 0,32 mm, film thickness 0,5 μm, Fa. Restek, Art.-Nr. 10339

### Run Sequence:

One Standard solution used for the system suitability test was injected five times at the beginning of each run. Other, separately prepared Standard solutions were injected twice in total with one injection following every three Sample solution injections. From each individual HPMC sample, three Sample solution preparations were made and one injection was done from each Sample solution. In each run, the same internal standard stock solution (n-octane in o-xylene) was used to prepare the Sample and Standard solutions.

### System Suitability:

The analysis run was valid if the relative standard deviation (RSD) of the relative peak areas for methyl iodide to n-octane (R_{Sa}) and isopropyl iodide to n-octane (R_{Sb}), calculated from five repeated injections of the same Standard solution, was not more than 1.0%.

### Calculations:

Weights were recorded to the nearest 0.01 mg. The average WₛₐiRₛₐ calculated from all Standard solution injections (W_{Sa} =weight of methyl iodide in standard solution) was used for calculating % methoxyl. The average W_{Sb}/R_{Sb} calculated from all Standard solution injections (W_{Sb}=weight of isopropyl iodide in the standard solution) was used for calculating % hydroxypropoxyl. The analysis run was valid if the relative standard deviation (RSD) of W_{Sa}/R_{Sa} and W_{Sb}/R_{Sb} calculated using all individual Standard solution injections was not more than 1.0%. The average of the calculated % methoxyl and % hydroxypropoxyl content of three sample solution preparations for each hypromellose sample was reported as the final result.
The values obtained were % methoxyl and % hydroxypropoxyl. These were subsequently converted into degree of substitution (DS) for methoxyl substituents and molar substitution (MS) for hydroxypropoxyl substituents. Residual amounts of salt have been taken into account in the conversion. The NaC1 content was 0.3 - 0.5% in all samples.

The viscosity of the HPMC samples was measured as a 2.0% by weight solution in water at 20° C. ±0.1° C. The 2.0% by weight HPMC solution in water was prepared according to United States Pharmacopeia (USP 35, “Hypromellose”, pages 3467-3469), followed by an Ubbelohde viscosity measurement according to DIN 51562-1:1999-01 (January 1999).

TABLE 1

| | (Comparative) Example | | | |
|---|---|---|---|---|
| | 1 | 2 | A | B |
| Viscosity at 20° C.¹⁾ | 3.4 | 4.3 | 3.1 | 3.1 |
| % methoxyl | 29.1 | 29.8 | 28.2 | 28.6 |
| % hydroxypropoxyl | 10.5 | 9.8 | 9.3 | 9.2 |
| Sum % methoxyl and hydroxypropoxyl | 39.6 | 39.6 | 37.5 | 37.8 |
| DS(methoxyl) | 1.94 | 1.98 | 1.85 | 1.88 |
| MS(hydroxypropoxyl) | 0.29 | 0.27 | 0.25 | 0.25 |

¹⁾measured as 2.0 weight percent solution in water

### Storage Stability

To evaluate the storage stability of a liquid composition of the present invention and of a comparative liquid composition, 10 weight percent of the HPMC of Examples 1 and 2 and of Comparative Examples A and B each were separately dissolved in a mixture of methanol/water having a weight ratio of 90/10 at room temperature for 2 hours.

The complex viscosity |η*| of the mixtures comprising the HPMC at 25° C. was investigated in a time sweep experiment using an Anton Paar Physica UDS200 rheometer (Ostfildern, Germany) in oscillation shear flow. A Cup & Bob (Z3-DIN) geometry was used and the upper surface of the geometry was covered with small metal sheets to avoid evaporation. The measurements were performed at a constant frequency of 1 Hz and a constant strain (deformation amplitude) of 0.5% over 18 h in the linear visco-elastic region. These measurements were conducted with a data collection rate of one average value each 5 minutes.

The results are summarized in Table 2 below. These results illustrate that a liquid composition of the present invention which comprises a HPMC that has 28 to 30 weight percent of methoxyl groups, 7 to 12 weight percent of hydroxypropoxyl groups and a sum of methoxyl groups and hydroxypropoxyl groups of from 38.5 to 42 weight percent is more storage stable and exhibits a smaller viscosity increase after storage of the liquid composition over an extended time period than a comparable liquid composition which comprises a HPMC which has 28 to 30 weight percent of methoxyl groups, 7 to 12 weight percent of hydroxypropoxyl groups and a sum of methoxyl groups and hydroxypropoxyl groups of less than 38.5 weight percent.

TABLE 2

| | 10 weight percent of HPMC dissolved in a mixture of | | | |
|---|---|---|---|---|
| | methanol/water of a weight ratio of 90/10 | | | |
| complex viscosity | | | Comparative | Comparative |
| \|η*\| | Example 1 | Example 2 | Example A | Example B |
| mPa · s at x min. | 1 | 2 | A | B |
| 5 | 2560 | 1200 | 394 | 462 |
| 30 | 3450 | 1190 | 429 | 564 |
| 60 | 3990 | 1180 | 559 | 615 |
| 120 | 4830 | 1190 | 611 | 752 |
| 180 | 5360 | 1190 | 657 | 1800 |
| 240 | 5730 | 1180 | 665 | 5330 |
| 300 | 6070 | 1190 | 699 | 14000 |
| 360 | 6430 | 1180 | 727 | 26800 |
| 420 | 6610 | 1190 | 733 | 39700 |
| 480 | 6840 | 1180 | 744 | 65600 |
| 540 | 6990 | 1190 | 777 | 115000 |
| 600 | 7200 | 1180 | 846 | 174000 |
| 660 | 7370 | 1170 | 2120 | 244000 |
| 720 | 7520 | 1180 | 51900 | 307000 |
| 780 | 7630 | 1190 | 1730000 | 370000 |
| 840 | 7760 | 1190 | 3660000 | 417000 |
| 900 | 7920 | 1170 | 4340000 | —* |
| 960 | 8030 | 1190 | 4970000 | —* |
| 1020 | 8130 | 1200 | 4650000 | —* |
| 1080 | 8200 | 1190 | 5870000 | —* |

*Defect in measurement occurred

1. A liquid composition comprising an organic liquid diluent and at least one hydroxypropyl methylcellulose having 28 to 30 weight percent of methoxyl groups, 7 to 12 weight percent of hydroxypropoxyl groups and a sum of methoxyl groups and hydroxypropoxyl groups of from 38.5 to 42 weight percent.

2. The liquid composition of claim 1 additionally comprising at least one active ingredient and optionally one or more adjuvants.

3. The liquid composition of claim 1 wherein the hydroxypropyl methylcellulose has a sum of methoxyl groups and hydroxypropoxyl groups of from 39.0 to 42 weight percent.

4. The liquid composition of claim 3 wherein the hydroxypropyl methylcellulose has a sum of methoxyl groups and hydroxypropoxyl groups of from 39.3 to 42 weight percent.

5. The liquid composition of claim 1 wherein the hydroxypropyl methylcellulose has 28.8 to 30 weight percent of methoxyl groups.

6. The liquid composition of claim 1 wherein the hydroxypropyl methylcellulose has a viscosity of from 1.20 to 100 mPa's, measured as a 2 wt.-% solution in water at 20° C.

7. The liquid composition of claim 2 wherein the hydroxypropyl methylcellulose has a viscosity of from 1.20 to 100 mPa's, measured as a 2 wt.-% solution in water at 20° C.

8. The liquid composition of claim 1 wherein the composition additionally comprises water and the composition comprises more than 50 weight percent of an organic liquid diluent and less than 50 weight percent of water, based on the total weight of organic liquid diluent and water.

9.-14. (canceled)

15. A process for coating a dosage form comprising the steps of
a) providing a liquid composition comprising an organic liquid diluent and at least one hydroxypropyl methylcellulose having 28 to 30 weight percent of methoxyl groups, 7 to 12 weight percent of hydroxypropoxyl groups and a sum of methoxyl groups and hydroxypropoxyl groups of from 38.5 to 42 weight percent, and
b) contacting the liquid composition with a dosage form.

16. The liquid composition of claim 4 wherein the hydroxypropyl methylcellulose has a sum of methoxyl groups and hydroxypropoxyl groups of from 39.5 to 41.5 weight percent.

17. The liquid composition of claim 5 wherein the hydroxypropyl methylcellulose has a sum of methoxyl groups and hydroxypropoxyl groups of from 39.5 to 41.5 weight percent.

18. The liquid composition of claim 1 comprising from 1 to 40 weight percent of said at least one hydroxypropyl methylcellulose, from 40 to 99 weight percent of an organic liquid and from 0 to 40 percent of an active ingredient, based on the total weight of the liquid composition.

19. The liquid composition of claim 17 comprising from 1 to 40 weight percent of said at least one hydroxypropyl methylcellulose, from 40 to 99 weight percent of an organic liquid and from 0 to 40 percent of an active ingredient, based on the total weight of the liquid composition.

20. The liquid composition of claim 8 comprising a blend of 80 to 95 weight percent of methanol and 20 to 5 weight percent of water, based on the total weight of organic liquid diluent and water.

21. The liquid composition of claim 20 wherein the hydroxypropyl methylcellulose has 28.8 to 30 weight percent of methoxyl groups and a sum of methoxyl groups and hydroxypropoxyl groups of from 39.5 to 41.5 weight percent.
